(19) United States
(12) Patent Application Publication
(10) Pub. No.: US 2020/0087453 A1
(43) Pub. Date: Mar. 19, 2020

(54) LOW SWELLING SYNTHETIC SEALANT

(71) Applicants: Baxter International Inc., Deerfield, IL (US); Baxter Healthcare SA, Glattpark (Opfikon) (CH)

(72) Inventor: Haitao Qian, Buffalo Grove, IL (US)

(21) Appl. No.: 16/574,338

(22) Filed: Sep. 18, 2019

Related U.S. Application Data
(60) Provisional application No. 62/733,293, filed on Sep. 19, 2018.

(51) Int. Cl. C08G 65/333; C08G 63/00; C08G 65/332; C08G 65/334; C08L 71/02; A61L 27/58; A61K 9/00

(52) U.S. Cl. CPC C08G 65/33389; C08G 63/00; C08G 65/3322; C08G 65/3342; C08L 2666/22; C08G 65/33337; A61L 27/58; A61K 9/0019; C08G 2650/58; C08L 71/02

(57) ABSTRACT

Compositions for use as a low-swelling tissue sealant are disclosed. The compositions include first and second precursor molecules, wherein the first precursor molecule is a polyoxyethylene homopolymer having at least two nucleophilic groups or a polyoxyethylene-polyoxypropylene block copolymer having at least two nucleophilic groups and the second precursor molecule is a polyoxyethylene-polyoxypropylene block copolymer having at least two electrophilic groups. Biomaterials for use as a low-swelling tissue sealant, methods of making such biomaterials, devices for applying a biomaterial to a tissue of a patient, methods of sealing a tissue of a patient, and kits also are disclosed.

### CROSS-REFERENCE TO RELATED APPLICATIONS

This application claims the benefit of U.S. Provisional Patent Application No. 62/733,293, filed Sep. 19, 2018, the entire content of which is hereby incorporated by reference in its entirety.

### BACKGROUND

### Field

The disclosure is directed to biomaterials useful as tissue glues and sealants. More particularly, the present disclosure is directed to biomaterials, precursor molecules for forming biomaterials, systems for using biomaterials, and methods of making and using biomaterials.

### Description of the Related Art

In the medical field, tissue glues and sealants are used to seal or repair biological tissue. A number of tissue glues and sealants are formed by mixing two or more components that react to form a biomaterial having sufficient strength and adhesion for a desired application, such as to seal or repair dura or other tissue. The separate sealant components are preferably biocompatible, and can be absorbed by the body, or are otherwise harmless to the body, so that they do not require later removal. For example, fibrin-based tissue sealants are made from a combination of at least two primary components, fibrinogen and thrombin. Upon coming into contact with each other, the fibrinogen and thrombin components interact to form a crosslinked polymeric network of fibrin that is suitable as a tissue sealant. Crosslinked gelatin-based tissue glues are another example of a two-component sealant and typically are formed by crosslinking gelatin with an aldehyde such as formaldehyde or glutaraldehyde. Another example of a tissue glue are urethane-based tissue glues that use a lysine diisocyanate ethyl ester to form a crosslinked biomaterial.

Other crosslinked polymer compositions include two-component systems based on polyethylene glycol and are disclosed, for example, in U.S. Pat. No. 5,874,500, which is hereby incorporated by reference in its entirety.

### SUMMARY

In light of the disclosure herein, and without limiting the scope of the invention in any way, in a first aspect of the present disclosure, which may be combined with any other aspect listed herein unless specified otherwise, a composition includes first and second precursor molecules wherein: i) the first precursor molecule has a formula I:

A-[(C₃H₆O)ₘ—(C₂H₄O)ₙ—B]_{y} (I)

wherein m is an integer from 0 to 200; n is an integer from 1 to 200; y is 2 or greater; A is a branch point; and B comprises a nucleophilic functional group; and ii) the second precursor molecule has a formula II:

D-[(C₃H₆O)ₚ—(C₂H₄O)_{q}-E]_{z} (II)

wherein p and q are independently integers from 1 to 200; z is 2 or greater; D is a branch point; and E comprises an electrophilic functional group substituted with a positively charged functional group, a negatively charged functional group, a zwitterionic functional group, a protonated or deprotonated version thereof, or a salt thereof.

In a second aspect of the present disclosure, which may be combined with any other aspect listed herein unless specified otherwise, a dual compartment syringe includes a composition as described herein loaded in a first compartment of the dual compartment syringe and a basic solution loaded in a second compartment of the dual compartment syringe.

In a third aspect of the present disclosure, which may be combined with any other aspect listed herein unless specified otherwise, the disclosure provides a biomaterial formed by reacting first and second precursor molecules wherein the first precursor molecule has a formula I as described herein, and the second precursor molecule has a formula II as described herein.

In a fourth aspect of the present disclosure, which may be combined with any other aspect listed herein unless specified otherwise, a biomaterial includes a crosslinked polymeric network having a structure:

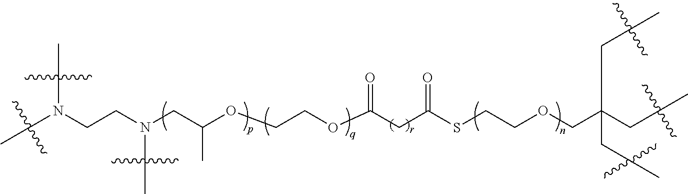

wherein n, p and q are independently integers from 1 to 200; and r is an integer from 1 to 5.

In a fifth aspect of the present disclosure, which may be combined with any other aspect listed herein unless specified otherwise, a biomaterial includes a crosslinked polymeric network having a structure:

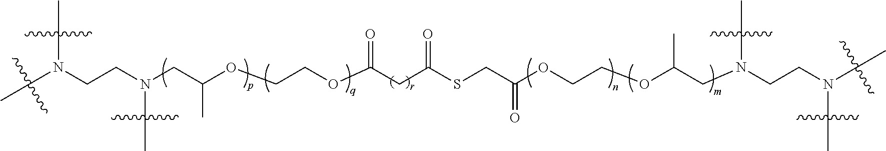

wherein m, n, p and q are independently integers from 1 to 200; and r is an integer from 1 to 5.

In a sixth aspect of the present disclosure, which may be combined with any other aspect listed herein unless specified otherwise, a method of making a biomaterial includes reacting first and second precursor molecules in the presence of a basic solution to form a biomaterial in the form of a crosslinked three dimensional network, wherein the first precursor molecule has a formula I as described herein, and the second precursor molecule has a formula II as described herein.

In a seventh aspect of the present disclosure, which may be combined with any other aspect listed herein unless specified otherwise, a method of sealing a tissue in a patient includes applying to a tissue in a patient a composition comprising first and second precursor molecules, wherein the first precursor molecule has a formula I as described herein, and the second precursor molecule has a formula II as described herein.

In a eighth aspect of the present disclosure, which may be combined with any other aspect listed herein unless specified otherwise, a kit includes a first precursor molecule and a second precursor molecule, wherein the first precursor molecule has a formula I as described herein, and the second precursor molecule has a formula II as described herein.

In light of the disclosure and aspects set forth herein, it is accordingly an advantage of the present disclosure to provide a biomaterial that is low-swelling and strong. Without wishing to be bound by theory, it is believed that by using a polyethylene oxide-polypropylene oxide block copolymer, the biomaterial is less swellable compared to a polyethylene oxide homopolymer having an otherwise similar number of monomer repeat units. Low-swelling tissue glues and sealants can be particularly advantageous in certain applications such as neural and spinal sealing.

It is another advantage of the present disclosure to provide biomaterial precursor molecules that have improved handling properties, including dissolving rapidly in aqueous solutions, being stable during long-term storage, and being stable during storage at ambient temperatures. Without wishing to be bound by theory, it is believed that precursor molecules having charged functional groups dissolve more rapidly in aqueous solutions than corresponding precursor molecules without such charged functional groups.

It is yet another advantage of the present disclosure to provide a synthetic biomaterial that does not contain proteins or animal products.

It is yet a further advantage of the present disclosure to provide a biomaterial that is partially or fully bioresorbable.

Additional features and advantages of the disclosed formulations are described in, and will be apparent from, the following Detailed Description. The features and advantages described herein are not all-inclusive and, in particular, many additional features and advantages will be apparent to one of ordinary skill in the art in view of the description. Also, any particular embodiment does not necessarily have to have all of the advantages listed herein. Moreover, it should be noted that the language used in the specification has been principally selected for readability and instructional purposes, and not to limit the scope of the inventive subject matter.

### DETAILED DESCRIPTION

Certain embodiments described herein relate generally to the field of tissue glues and sealants. More particularly, some embodiments described herein relate to compositions comprising first and second precursor molecules. Related embodiments described herein relate to dual compartment syringes comprising such first and second precursor molecules loaded in the compartments of the dual compartment syringe. Additional related embodiments described herein relate to biomaterials formed by reaction of such first and second precursor molecules. Further related embodiments described herein relate to biomaterials comprising a crosslinked polymeric network. Related embodiments described herein also relate to methods of making biomaterials by reacting such first and second precursor molecules. Additional related embodiments described herein relate to methods of sealing a tissue in a patient by applying compositions comprising such first and second precursor molecules to the tissue in the patient. Further related embodiments described herein relate to kits comprising such first and second precursor molecules.

As used herein, the term “substantially free” may refer to a composition that contains no more than 1% of the specified component, for example, no more than 0.5%, no more than 0.1%, no more than 0.05%, no more than 0.02%, no more than 0.01%, no more than 0.005%, no more than 0.002%, and/or no more than 0.001% of the specified component. For example, a composition that is “substantially free of poly(ethylene oxide) derivatives” may refer to a composition that contains no more than 1% poly(ethylene oxide) derivatives, such as no more than 0.5%, no more than 0.1%, no more than 0.05%, no more than 0.02%, no more than 0.01%, no more than 0.005%, no more than 0.002%, and/or no more than 0.001% poly(ethylene oxide) derivatives.

As used herein, the term “poly(ethylene oxide) derivative” refers to a straight-chain or branched poly(ethylene oxide) homopolymer modified to contain one or more (e.g., two, three, four, six, or eight) nucleophilic or electrophilic groups.

### Compositions

The disclosure provides compositions for the manufacture of a biomaterial suitable for use as a tissue glue or sealant. The compositions contain at least first and second precursor molecules that are multifunctional (e.g., di-, tri-, or tetra-functional) for forming crosslinked networks, such as crosslinked polymeric networks. The precursor molecules can crosslink in situ at the site of need in a human or animal to form the polymeric network of the biomaterial.

The precursor molecules disclosed herein include a first precursor molecule containing at least two nucleophilic functional groups and a second precursor molecule containing at least two electrophilic functional groups. The nucleophilic groups of the first precursor molecule and the electrophilic groups of the second precursor molecule are selected such that the nucleophilic groups are capable of reacting with the electrophilic groups to form covalent linkages between the first and second precursor molecules, typically under physiological conditions or under basic conditions. This can be achieved by different reaction mechanisms, including a nucleophilic substitution reaction.

The first precursor molecules disclosed herein are polyethylene oxide homopolymers and polypropylene oxide-polyethylene oxide (PPO-PEO) block copolymers that contain at least two nucleophilic functional groups. In an embodiment of the present disclosure, which may be combined with any other embodiment listed herein unless specified otherwise, the first precursor molecule has a formula I:

A-[(C₃H₆O)ₘ—(C₂H₄O)ₙ—B]_{y} (I)

In an embodiment of the present disclosure, which may be combined with any other embodiment listed herein unless specified otherwise, m of formula I is an integer from 0 to 200, such as 0, from 1 to 200, from 2 to 200, from 4 to 100, from 5 to 50, from 6 to 45, from 8 to 40, from 10 to 35, from 12 to 30, from 15 to 25, from 16 to 20, and/or 18. In an embodiment of the present disclosure, which may be combined with any other embodiment listed herein unless specified otherwise, n of formula I is an integer from 1 to 200, such as from 2 to 200, from 5 to 100, from 10 to 90, from 20 to 80, from 30 to 75, from 40 to 70, from 45 to 65, from 50 to 60, and/or 56.

In an embodiment of the present disclosure, which may be combined with any other embodiment listed herein unless specified otherwise, y of formulae I is 2 or greater, such as 2 to 8, 2 to 6, 2 to 4, 2, 3, or 4.

In an embodiment of the present disclosure, which may be combined with any other embodiment listed herein unless specified otherwise, A of formula I is a branch point. In an embodiment of the present disclosure, which may be combined with any other embodiment listed herein unless specified otherwise, branch point A is selected from the group consisting of carbon, glycerol, pentaerythritol, dipentaerythritol, and ethylene diamine.

In an embodiment of the present disclosure, which may be combined with any other embodiment listed herein unless specified otherwise, B of formula I comprises a nucleophilic functional group. In an embodiment of the present disclosure, which may be combined with any other embodiment listed herein unless specified otherwise, B comprises a thiol group or an amino group.

The first precursor molecules are multifunctional monomers, oligomers, and/or polymers. In an embodiment of the present disclosure, which may be combined with any other embodiment listed herein unless specified otherwise, the first precursor molecule has a number average molecular weight of about 2 kD to about 10 kD, such as about 2 kD to about 8 kD, about 3 kD to about 7 kD, about 4 kD to about 6 kD, about 5 kD, about 2 kD to about 20 kD, about 5 kD to about 20 kD, about 10 kD to about 15 kD, about 15 kD to about 20 kD, and/or about 12.5 kD to about 17.5 kD.

In an embodiment of the present disclosure, which may be combined with any other embodiment listed herein unless specified otherwise, the first precursor molecule is a 4-arm polyethylene oxide homopolymer of formula I, wherein A is a pentaerythritol molecule and B comprises a thiol group, such as a (CH₂)₂SH group. Four-arm polyethylene oxide homopolymers with a pentaerythritol core molecule are sold, for example, by Sigma-Aldrich Chemical Co.

In an embodiment of the present disclosure, which may be combined with any other embodiment listed herein unless specified otherwise, the first precursor molecule has a formula:

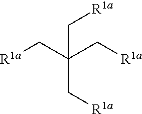

wherein each R^{1a} is

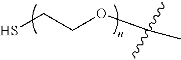

n is an integer from 1 to 200, such as 10 to 200, 20 to 100, and/or 30 to 70.

In an embodiment of the present disclosure, which may be combined with any other embodiment listed herein unless specified otherwise, the first precursor molecule is a 4-arm poly(ethylene oxide-polypropylene oxide) block copolymer of formula I, wherein A is an ethylene diamine molecule and B comprises a thiol group, such as a COCH₂SH group. Four-arm poly(ethylene oxide-polypropylene oxide) block copolymers with an ethylene diamine core molecule are sold, for example, by BASF under the tradename TETRONIC.

In an embodiment of the present disclosure, which may be combined with any other embodiment listed herein unless specified otherwise, the first precursor molecule has a formula:

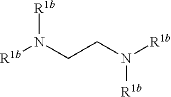

wherein each R^{1b} is

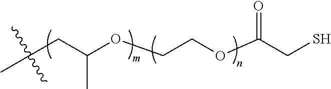

m is an integer from 1 to 200, such as from 2 to 200, from 4 to 100, from 5 to 50, from 6 to 45, from 8 to 40, from 10 to 35, from 12 to 30, from 15 to 25, from 16 to 20, and/or 18; and n is an integer from 1 to 200, such as from 2 to 200, from 5 to 100, from 10 to 90, from 20 to 80, from 30 to 75, from 40 to 70, from 45 to 65, from 50 to 60, and/or 56.

The second precursor molecules disclosed herein are polypropylene oxide-polyethylene oxide (PPO-PEO) block copolymers that contain at least two electrophilic functional groups. In an embodiment of the present disclosure, which may be combined with any other embodiment listed herein unless specified otherwise, the second precursor molecule has a formula II:

D-[(C₃H₆O)ₚ—(C₂H₄O)_{q}-E]_{z} (II)

In an embodiment of the present disclosure, which may be combined with any other embodiment listed herein unless specified otherwise, p is an integer from 1 to 200, such as from 2 to 200, from 4 to 100, from 5 to 50, from 6 to 45, from 8 to 40, from 10 to 35, from 12 to 30, from 15 to 25, from 16 to 20, and/or 18; and q is an integer from 1 to 200, such as from 2 to 200, from 5 to 100, from 10 to 90, from 20 to 80, from 30 to 75, from 40 to 70, from 45 to 65, from 50 to 60, and/or 54.

In an embodiment of the present disclosure, which may be combined with any other embodiment listed herein unless specified otherwise, z of formula II is 2 or greater, such as 2 to 8, 2 to 6, 2 to 4, 2, 3, or 4.

In an embodiment of the present disclosure, which may be combined with any other embodiment listed herein unless specified otherwise, D of formula II is a branch point. In an embodiment of the present disclosure, which may be combined with any other embodiment listed herein unless specified otherwise, branch point D is selected from the group consisting of carbon, glycerol, pentaerythritol, dipentaerythritol, and ethylene diamine.

In an embodiment of the present disclosure, which may be combined with any other embodiment listed herein unless specified otherwise, E of formula II comprises an electrophilic functional group substituted with a positively charged functional group, a negatively charged functional group, a zwitterionic functional group, a protonated or deprotonated version thereof, or a salt thereof. Suitable electrophilic groups include, but are not limited to, mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters (e.g., succinimidyl carbonate, succinimidyl glutarate, succinimidyl acetate, succinimidyl succinimide), N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; isocyanates; maleimides; substituted maleimides; haloalkanes; epoxides; imines; aziridines; olefins (including conjugated olefins) such as vinyl sulfone, ethenesulfonyl, etheneimino, acrylate, methacrylate, and α,β-unsaturated aldehydes and ketones; and combinations thereof. In an embodiment of the present disclosure, which may be combined with any other embodiment listed herein unless specified otherwise, the electrophilic functional group comprises a succinimidyl ester group.

The electrophilic functional groups are substituted with a charged functional group or a functional group capable of becoming charged by protonation or deprotonation. Without wishing to be bound by theory, it is believed that including a charged functional group or a group capable of becoming charged improves the aqueous solubility and handling properties of the second precursor molecule. The charged functional group may be positively charged (or capable of becoming positively charged by protonation), negatively charged (or capable of becoming negatively charged by deprotonation), or zwitterionic. Functional groups that are capable of becoming charged by being protonated or deprotonated typically become charged in a pH-dependent manner. For example, functional groups capable of becoming negatively charged by deprotonation typically become negatively charged at high pH and functional groups capable of becoming positively charged by protonation typically become positively charged at low pH.

The substituent groups for the electrophilic functional groups include negatively charged functional groups, protonated versions thereof, or salts thereof, such as sulfonic acids, sulfonates, sulfinic acids, sulfinates, carboxylic acids, carboxylates, phosphonic acids, phosphonates, phosphoric acids, and phosphates In an embodiment of the present disclosure, which may be combined with any other embodiment listed herein unless specified otherwise, E comprises an electrophilic functional group substituted with an acid moiety or an anion or salt thereof. In an embodiment of the present disclosure, which may be combined with any other embodiment listed herein unless specified otherwise, the acid moiety or anion or salt thereof is selected from SO₃H, SO₃⁻, SO₂H, SO₂⁻, CO₂H, CO₂⁻, PO₃H₂, PO₃⁻, OPO₃H₂, OPO₃⁻², or a salt thereof.

The substituent groups for the electrophilic functional groups include positively charged functional groups, deprotonated versions thereof, or salts thereof, such as amino groups, quaternary ammonium groups, and phosphonium groups.

The substituent groups for the electrophilic functional groups include zwitterionic functional groups, protonated or deprotonated versions thereof, or salts thereof, such as amino acids, sulfamic acids, phosphatidylcholines, and betaines.

In an embodiment of the present disclosure, which may be combined with any other embodiment listed herein unless specified otherwise, E comprises a sulfosuccinimidyl ester group.

In an embodiment of the present disclosure, which may be combined with any other embodiment listed herein unless specified otherwise, E is:

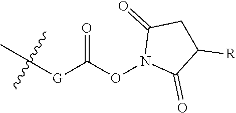

wherein G is absent or —CO(CH₂)ᵣ—; r is an integer from 1 to 5; and R is a positively charged functional group, a negatively charged functional group, a zwitterionic functional group, a protonated or deprotonated version thereof, or a salt thereof. In an embodiment of the present disclosure, which may be combined with any other embodiment listed herein unless specified otherwise, E is:

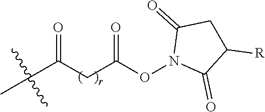

wherein r is an integer from 1 to 5; and R is a positively charged functional group, a negatively charged functional group, a zwitterionic functional group, a protonated or deprotonated version thereof, or a salt thereof. In an embodiment of the present disclosure, which may be combined with any other embodiment listed herein unless specified otherwise, r is an integer from 2 to 4, such as 3. In an embodiment of the present disclosure, which may be combined with any other embodiment listed herein unless specified otherwise, R is an acid moiety or an anion or salt thereof. In an embodiment of the present disclosure, which may be combined with any other embodiment listed herein unless specified otherwise, the acid moiety or anion or salt thereof is selected from the group consisting of SO₃H, SO₃⁻, SO₂H, SO₂⁻, CO₂H, CO₂⁻, PO₃H₂, PO₃⁻, OPO₃H₂, OPO₃⁻², and salts thereof. In an embodiment of the present disclosure, which may be combined with any other embodiment listed herein unless specified otherwise, r is 3 and R is SO₃H, SO₃⁻, or a salt thereof.

The second precursor molecules are multifunctional monomers, oligomers, and/or polymers. In an embodiment of the present disclosure, which may be combined with any other embodiment listed herein unless specified otherwise, the second precursor molecule has a number average molecular weight of about 2 kD to about 20 kD, such as about 5 kD to about 20 kD, about 10 kD to about 15 kD, about 15 kD to about 20 kD, and/or about 12.5 kD to about 17.5 kD.

In an embodiment of the present disclosure, which may be combined with any other embodiment listed herein unless specified otherwise, the second precursor molecule is a 4-arm poly(ethylene oxide-polypropylene oxide) block copolymer of formula II, wherein D is an ethylene diamine molecule and E comprises a succinimidyl group, such as a

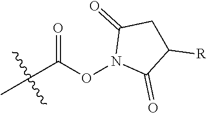

group or

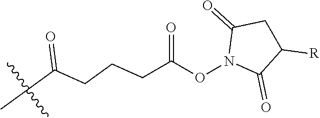

group, wherein R is as defined herein. Four-arm poly(ethylene oxide-polypropylene oxide) block copolymers with an ethylene diamine core molecule are sold, for example, by BASF under the tradename TETRONIC.

In an embodiment of the present disclosure, which may be combined with any other embodiment listed herein unless specified otherwise, the second precursor molecule has a formula:

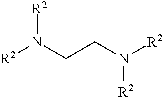

wherein each R² is

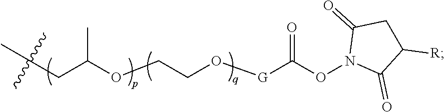

p is an integer from 1 to 200, such as from 2 to 200, from 4 to 100, from 5 to 50, from 6 to 45, from 8 to 40, from 10 to 35, from 12 to 30, from 15 to 25, from 16 to 20, and/or 18; q is an integer from 1 to 200, such as from 2 to 200, from 5 to 100, from 10 to 90, from 20 to 80, from 30 to 75, from 40 to 70, from 45 to 65, from 50 to 60, and/or 54; G is absent or —CO(CH₂)ᵣ—; r is an integer from 1 to 5; and R is a positively charged functional group, a negatively charged functional group, a zwitterionic functional group, a protonated or deprotonated version thereof, or a salt thereof. In an embodiment of the present disclosure, which may be combined with any other embodiment listed herein unless specified otherwise, the second precursor molecule has a formula:

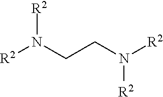

wherein each R² is

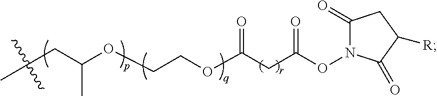

p is an integer from 1 to 200, such as from 2 to 200, from 4 to 100, from 5 to 50, from 6 to 45, from 8 to 40, from 10 to 35, from 12 to 30, from 15 to 25, from 16 to 20, and/or 18; q is an integer from 1 to 200, such as from 2 to 200, from 5 to 100, from 10 to 90, from 20 to 80, from 30 to 75, from 40 to 70, from 45 to 65, from 50 to 60, and/or 54; r is an integer from 1 to 5; and R is a positively charged functional group, a negatively charged functional group, a zwitterionic functional group, a protonated or deprotonated version thereof, or a salt thereof. In an embodiment of the present disclosure, which may be combined with any other embodiment listed herein unless specified otherwise, r is an integer from 2 to 4, such as 3. In an embodiment of the present disclosure, which may be combined with any other embodiment listed herein unless specified otherwise, R is an acid moiety or an anion or salt thereof. In an embodiment of the present disclosure, which may be combined with any other embodiment listed herein unless specified otherwise, the acid moiety or anion or salt thereof is selected from the group consisting of SO₃H, SO₃⁻, SO₂H, SO₂⁻, CO₂H, CO₂⁻, PO₃H₂, PO₃⁻, OPO₃H₂, OPO₃⁻², and salts thereof. In an embodiment of the present disclosure, which may be combined with any other embodiment listed herein unless specified otherwise, r is 3 and R is SO₃H, SO₃⁻, or a salt thereof.

The first and second precursor molecules are typically provided in the compositions described herein in a ratio such that a majority of the functional groups of both components react with their respective counterparts. In an embodiment of the present disclosure, which may be combined with any other embodiment listed herein unless specified otherwise, the ratio of functional groups of the first precursor molecule to functional groups of the second precursor molecule (i.e., the ratio of nucleophilic functional groups to electrophilic functional groups) is in the range of about 0.7 to about 1.3, such as about 0.8 to about 1.2, about 0.9 to about 1.1, and/or about 1 to 1.

The nucleophilic groups of the first precursor molecule and the electrophilic groups of the second precursor molecule typically do not react with each other under acidic conditions. This allows the first and second precursor molecules to be provided together in a composition that can be activated for reaction at a desired time by increasing the pH to physiological or basic pH. In an embodiment of the present disclosure, which may be combined with any other embodiment listed herein unless specified otherwise, the composition comprising the first and second precursor molecules has a pH of about 3 or less, such as about 1 to about 3, about 1.5 to about 2.5, about 1.6 to about 2.4, about 1.7 to about 2.3, about 1.8 to about 2.2, about 1.9 to about 2.1, and/or about 2.

The nucleophilic groups of the first precursor molecule and the electrophilic groups of the second precursor molecule typically react with each other under physiological conditions or under basic conditions. In an embodiment of the present disclosure, which may be combined with any other embodiment listed herein unless specified otherwise, the composition comprising the first and second precursor molecules further comprises a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the composition comprising first and second precursor molecules as described herein has a pH of about 7 to about 12, such as about 8 to about 11, about 9 to about 10, about 7 to about 8, about 8 to about 9, about 9 to about 10, and/or about 10 to about 11.

In an embodiment of the present disclosure, which may be combined with any other embodiment listed herein unless specified otherwise, the composition comprises a first precursor molecule that is a polyoxyethylene-polyoxypropylene block copolymer and a second precursor molecule that is a polyoxyethylene-polyoxypropylene block copolymer, and is free or substantially free of poly(ethylene oxide) derivatives.

### Devices

The disclosure provides devices to apply a biomaterial to a human or animal for use as a tissue glue or sealant. The devices comprise first and second precursor molecules as described herein, particularly first precursor molecules of formula I, and second precursor molecules of formulae II as described in the foregoing “Compositions” section. In an embodiment of the present disclosure, which may be combined with any other embodiment listed herein unless specified otherwise, a dual compartment syringe comprises the composition comprising the first and second precursor molecules loaded in a first compartment of the dual compartment syringe and a basic solution loaded in a second compartment of the dual compartment syringe. A suitable dual compartment syringe typically includes first and second compartments (or barrels), a plunger, and optionally a syringe cap. An example of a dual compartment syringe device is described in U.S. Pat. No. 4,359,049, which is hereby incorporated by reference in its entirety.

The nucleophilic groups of the first precursor molecule and the electrophilic groups of the second precursor molecule typically react with each other under physiological conditions or under basic conditions. In an embodiment of the present disclosure, which may be combined with any other embodiment listed herein unless specified otherwise, the second compartment includes a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the second compartment includes a weak base, such as carbonate, N-methyldiethanolamine, 3-dimethylamino-1-propanol, and/or 2-dimethylamino-ethanol. In an embodiment of the present disclosure, which may be combined with any other embodiment listed herein unless specified otherwise, the basic solution has a pH of about 7 to about 12, such as about 8 to about 11, about 8.5 to about 10.5, about 9 to about 10, about 9.2 to about 10, about 9.4 to about 9.8, about 9.5 to about 9.7, and/or about 9.6. In an embodiment of the present disclosure, which may be combined with any other embodiment listed herein unless specified otherwise, the basic solution comprises carbonate, phosphate, or a mixture thereof.

In an embodiment of the present disclosure, which may be combined with any other embodiment listed herein unless specified otherwise, the first and/or second compartments are free or substantially free of poly(ethylene oxide) derivatives.

In an embodiment of the present disclosure, which may be combined with any other embodiment listed herein unless specified otherwise, the dual compartment syringe is connected to a mixing device such as a static mixer for mixing the first precursor molecule with the second precursor molecule. The first and second precursors molecule can then be extruded into the mixing device, while the basic solution is simultaneously extruded into the mixing device. Within the mixing device, the first and the second precursor molecules are mixed together with the basic solution to thereby form a reactive mixture. Thereafter, the reactive mixture can be extruded through an orifice and onto a surface (e.g., tissue), where a biomaterial is formed, which can function as a tissue glue or sealant, or the like. The reactive mixture begins forming a three-dimensional matrix immediately upon being formed by the mixing of the first and second precursor molecules and the basic solution in the mixing device. Accordingly, the reactive mixture is preferably extruded from the mixing device onto the tissue very quickly after it is formed so that the three-dimensional matrix forms on, and is able to adhere to, the tissue.

### Biomaterials

The disclosure provides biomaterials for use as a tissue glue or sealant in a human or animal. In an embodiment of the present disclosure, which may be combined with any other embodiment listed herein unless specified otherwise, the disclosure provides a biomaterial formed by reacting first and second precursor molecules as described herein, particularly first precursor molecules of formula I and second precursor molecules of formula II as described in the foregoing “Compositions” section.

In an embodiment of the present disclosure, which may be combined with any other embodiment listed herein unless specified otherwise, a biomaterial is provided comprising a crosslinked polymeric network having a structure:

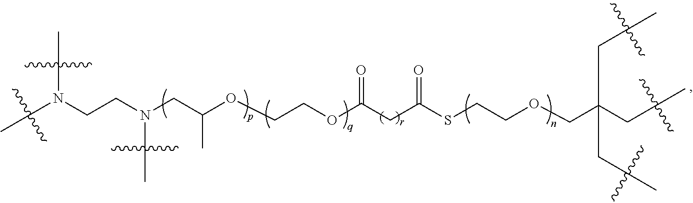

wherein n, p and q are independently integers from 1 to 200, including any of the ranges and values described in the foregoing “Compositions” section; and r is an integer from 1 to 5. In the formulae above, use of the symbol [symbol] indicates that the molecular structure beyond this point is unspecified crosslinked material generally formed by reaction of the first and second precursor molecules as described herein. In an embodiment of the present disclosure, which may be combined with any other embodiment listed herein unless specified otherwise, r is an integer from 2 to 4, such as 3.

In an embodiment of the present disclosure, which may be combined with any other embodiment listed herein unless specified otherwise, a biomaterial is provided comprising a crosslinked polymeric network having a structure:

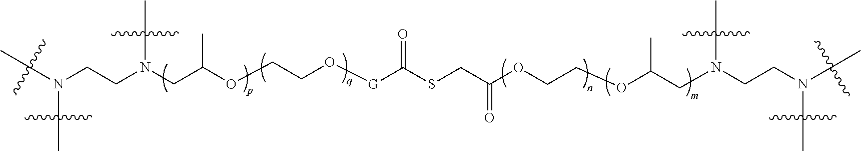

wherein m, n, p and q are independently integers from 1 to 200, including any of the ranges and values described in the foregoing “Compositions” section; G is absent or —CO(CH₂)ᵣ—; and r is an integer from 1 to 5. In an embodiment of the present disclosure, which may be combined with any other embodiment listed herein unless specified otherwise, a biomaterial is provided comprising a crosslinked polymeric network having a structure:

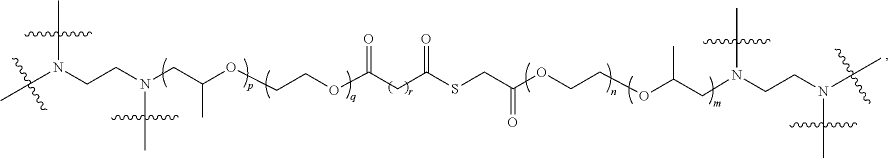

wherein m, n, p and q are independently integers from 1 to 200, including any of the ranges and values described in the foregoing “Compositions” section; and r is an integer from 1 to 5. In the formulae above, use of the symbol [symbol] indicates that the molecular structure beyond this point is unspecified crosslinked material generally formed by reaction of the first and second precursor molecules as described herein. In an embodiment of the present disclosure, which may be combined with any other embodiment listed herein unless specified otherwise, r is an integer from 2 to 4, such as 3.

In an embodiment of the present disclosure, which may be combined with any other embodiment listed herein unless specified otherwise, the disclosure provides a method of making a biomaterial comprising reacting first and second precursor molecules as described herein, particularly first precursor molecules of formula I and second precursor molecules of formula II as described in the foregoing “Compositions” section to form a biomaterial in the form of a crosslinked three dimensional network. In an embodiment of the present disclosure, which may be combined with any other embodiment listed herein unless specified otherwise, the method comprises reacting the first and second precursor molecules in the presence of a basic solution, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the first and second precursor molecules are dissolved in an acidic aqueous solution having a pH of 3 or less before the reacting step. In an embodiment of the present disclosure, which may be combined with any other embodiment listed herein unless specified otherwise, the method comprises reacting the first and second precursor molecules under basic conditions, such as at a pH of about 7 to about 12, about 8 to about 11, about 9 to about 10, about 7 to about 8, about 8 to about 9, about 9 to about 10, and/or about 10 to about 11.

### Methods of Use

The compositions disclosed herein can be used in a variety of different applications. In general, these compositions can be adapted for use in any tissue engineering application where synthetic gel matrices are currently being utilized. For example, the compositions are useful as tissue glues and sealants, vascular sealants, in tissue augmentation, in tissue repair, as hemostatic agents, and in preventing tissue adhesions, and may be used in a variety of open, endoscopic, and laparoscopic surgical procedures. One of skill in the art can easily determine the appropriate administration protocol to use with any particular composition having a known gel strength and gelation time.

In one application, the compositions described herein can be used for medical conditions that require a coating or sealing layer to prevent the leakage of gases, liquid or solids.

Methods of use typically entail applying the composition to the damaged tissue or organ to seal 1) vascular and or other tissues or organs to stop or minimize the flow of blood; 2) thoracic tissue to stop or minimize the leakage of air; 3) gastrointestinal tract or pancreatic tissue to stop or minimize the leakage of fecal or tissue contents; 4) bladder or urethra to stop or minimize the leakage of urine; 5) dura to stop or minimize the leakage of cerebrospinal fluid; and/or 6) skin or serosal tissue to stop the leakage of serosal fluid. These compositions may also be used to adhere tissues together such as small vessels, nerves or dermal tissue. The compositions can be used 1) by applying them to the surface of one tissue and then a second tissue may be rapidly pressed against the first tissue or 2) by bringing the tissues in close juxtaposition and then applying the compositions such that both the first and second tissues are contacted with the compositions. In addition, the compositions can be used to fill spaces in soft and hard tissues that are created by disease or surgery. The compositions may additionally be used for anchoring or affixing surgical implants, such as in prosthetic fixation to, e.g., fix a mesh to tissue during hernia repair.

In an embodiment of the present disclosure, which may be combined with any other embodiment listed herein unless specified otherwise, the disclosure provides a method of sealing a tissue in a patient comprising applying to a tissue in a patient a composition comprising first and second precursor molecules as described herein, particularly first precursor molecules of formula I and second precursor molecules of formula II as described in the foregoing “Compositions” section.

### Kits

The disclosure provides kits for making a biomaterial for use as a tissue glue or sealant. Precursor molecules can be packaged in kits, typically along with written or otherwise illustrated instructions for use. The kits comprise first and second precursor molecules as described herein, particularly first precursor molecules of formula I and second precursor molecules of formula II as described in the foregoing “Compositions” section. In an embodiment of the present disclosure, which may be combined with any other embodiment listed herein unless specified otherwise, a kit comprises the first precursor molecule and the second precursor molecule. In an embodiment of the present disclosure, which may be combined with any other embodiment listed herein unless specified otherwise, the kit further comprises a basic solution. In an embodiment of the present disclosure, which may be combined with any other embodiment listed herein unless specified otherwise, the basic solution comprises carbonate, phosphate, or a mixture thereof. Typically, each component of the kit is packaged separately such that the component remains sterile and does not contact another component. For example, kits comprising a dual compartment syringe and at least one applicator may include a first sterile packaging containing the dual compartment syringe able to be delivered to the sterile field as well as a second sterile packaging containing the applicator, optionally with a static mixer.

In an embodiment of the present disclosure, which may be combined with any other embodiment listed herein unless specified otherwise, the kit comprises a dual compartment syringe as described herein. In an embodiment of the present disclosure, which may be combined with any other embodiment listed herein unless specified otherwise, the first and second precursor molecules are present in a first compartment of the dual compartment syringe and a basic solution is present in a second compartment of the dual compartment syringe.

In an embodiment of the present disclosure, which may be combined with any other embodiment listed herein unless specified otherwise, the first precursor molecule is present in a first container and the second precursor molecule is present in a second container. In an embodiment of the present disclosure, which may be combined with any other embodiment listed herein unless specified otherwise, the first and second molecules are present in the same container.

The nucleophilic groups of the first precursor molecule and the electrophilic groups of the second precursor molecule typically react with each other under physiological conditions or under basic conditions. In an embodiment of the present disclosure, which may be combined with any other embodiment listed herein unless specified otherwise, the second container includes a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the second container includes a weak base, such as carbonate, N-methyldiethanolamine, 3-dimethylamino-1-propanol, and/or 2-dimethylamino-ethanol. In an embodiment of the present disclosure, which may be combined with any other embodiment listed herein unless specified otherwise, the basic solution has a pH of about 7 to about 12, such as about 8 to about 11, about 8.5 to about 10.5, about 9 to about 10, about 9.2 to about 10, about 9.4 to about 9.8, about 9.5 to about 9.7, and/or about 9.6. In an embodiment of the present disclosure, which may be combined with any other embodiment listed herein unless specified otherwise, the basic solution comprises carbonate, phosphate, or a mixture thereof.

In an embodiment of the present disclosure, which may be combined with any other embodiment listed herein unless specified otherwise, the first and second precursor molecules are present in the kit in liquid form. In an embodiment of the present disclosure, which may be combined with any other embodiment listed herein unless specified otherwise, the first and second precursor molecules are present in the kit in powder form.

In an embodiment of the present disclosure, which may be combined with any other embodiment listed herein unless specified otherwise, the first and/or second containers are free or substantially free of poly(ethylene oxide) derivatives.

In an embodiment of the present disclosure, which may be combined with any other embodiment listed herein unless specified otherwise, a single compartment syringe contains the first precursor molecules of formula I and the second precursor molecules of formula II as described in the foregoing “Compositions” section. In an embodiment of the present disclosure, which may be combined with any other embodiment listed herein unless specified otherwise, the first and second precursor molecules are present in the single compartment syringe in powder form. In an embodiment of the present disclosure, which may be combined with any other embodiment listed herein unless specified otherwise, the first and second precursor molecules are present in the single compartment syringe in liquid form. In an embodiment of the present disclosure, which may be combined with any other embodiment listed herein unless specified otherwise, the single compartment syringe is provided with a dual compartment syringe containing an acidic solution or water in a first (acidic) compartment of the dual compartment syringe, and a basic solution as described herein in a second (basic) compartment of the dual compartment syringe. To prepare a sealant composition, the contents of the single compartment syringe may be transferred to the first (acidic) compartment of the dual compartment syringe using a transfer port to be dissolved or diluted to an acidic pH. Typically, once the first and second precursor molecules are added to the first (acidic) compartment of the dual compartment syringe, the composition comprising the first and second precursor molecules in the first (acidic) chamber of the dual compartment syringe has a pH of about 3 or less, such as about 1 to about 3, about 1.5 to about 2.5, about 1.6 to about 2.4, about 1.7 to about 2.3, about 1.8 to about 2.2, about 1.9 to about 2.1, and/or about 2. In an embodiment of the present disclosure, which may be combined with any other embodiment listed herein unless specified otherwise, the single compartment syringe is provided with at least one applicator, optionally with a mixing device.

Suitable kits are not limited to the devices described herein and may also include any other suitable delivery device known in the art.

### EXAMPLES

### Example 1

### Synthesis of Sulfo-NHS Glutarate of Ethylenediamine Tetrakis(Ethoxylate-Block-Propoxylate) Tetrol

TETRONIC 1107 sulfo-NHS glutarate was prepared as shown in Scheme 1 using TETRONIC 1107 ethylenediamine tetrakis(ethoxylate-block-propoxylate) tetrol, average Mₙ 15,000, as the starting material. In the first step, 90.00 g of TETRONIC 1107 and 300 ml of toluene were refluxed in a 500 ml flask under argon protection using a Dean-Stark distillation apparatus to collect the azeotropic water. After 4 hours of distillation, the volume was concentrated to 250 ml and allowed to cool before adding 3.30 g of glutaric anhydride, 303 mg of 4-dimethylaminopyridien (DMAP), and 3.57 g of triethylamine (TEA). The resulting mixture was stirred at room temperature overnight and then the solution was poured into 1.4 liter of methyl tert-butyl ether (MTBE) while stirring. The mixture was then filtered and the solid was washed with MTBE 4 times (200 ml each). The obtained solid was charged in a 500 ml flask containing 200 ml of toluene and heated at 30° C. to dissolve the solid. The solution was next concentrated to about 200 ml under vacuum at 30° C. and then freeze-dried to yield TETRONIC 1107 glutarate.

In the second step, 4.560 g of TETRONIC 1107 glutarate was added to 7.9 g of DMSO-d6, 346 mg of sulfo-NHS was added to the mixture, and the mixture was stirred until most of the polymer was dissolved. Next, 300 microliter of N,N′-diisopropylcarbodiimide (DIC) was added and the reaction was stirred. After the reaction was complete, the solution was poured into 500 ml of MTBE, the resulting mixture was filtered, and the solid washed with MTBE. The obtained solid was dissolved in 200 ml of dichloromethane, washed with sodium bicarbonate solution, and the aqueous phase was separated from the organic phase. The resulting organic phase was washed with brine three times (80 ml each) and then dried over sodium sulfate. After filtration and concentration under vacuum, the solution was poured into MTBE, the mixture was filtered, washed with MTBE, and then dried under vacuum to yield 1.83 g TETRONIC 1107 sulfo-NHS glutarate. Advantageously, 92% or higher conversion can be obtained by coupling sulfo-NHS with TETRONIC 1107 glutarate in dimethylsulfoxide (DMSO), compared to conversion of around 60% in dimethylformamide (DMF) or dimethylacetamide (DMA).

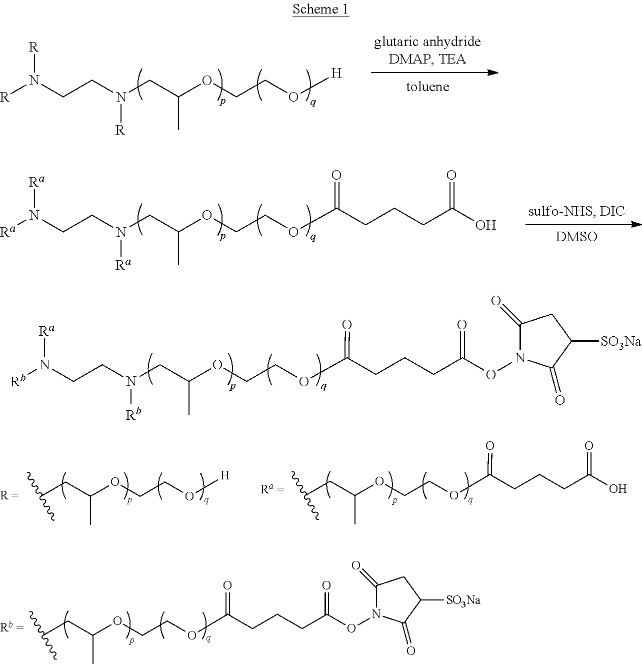

### Example 2

### Preparation of Low Swelling Synthetic Sealant

A crosslinked synthetic tissue sealant was prepared as shown in Scheme 2 by reacting the TETRONIC 1107 sulfo-NHS glutarate prepared in Example 1 with a 4-arm thiol-substituted polyethylene glycol. In particular, TETRONIC 1107 sulfo-NHS glutarate and 4-arm-PEG-SH (average Mₙ˜10,000) were dissolved in pH 2 hydrochloric acid solution and then placed in a first syringe barrel of a dual syringe device. A solution of sodium carbonate buffered with phosphate (pH 9.6) was placed in the second syringe barrel of the dual syringe device. The two solutions were then co-sprayed using the dual syringe device to form a low-swelling tissue sealant.

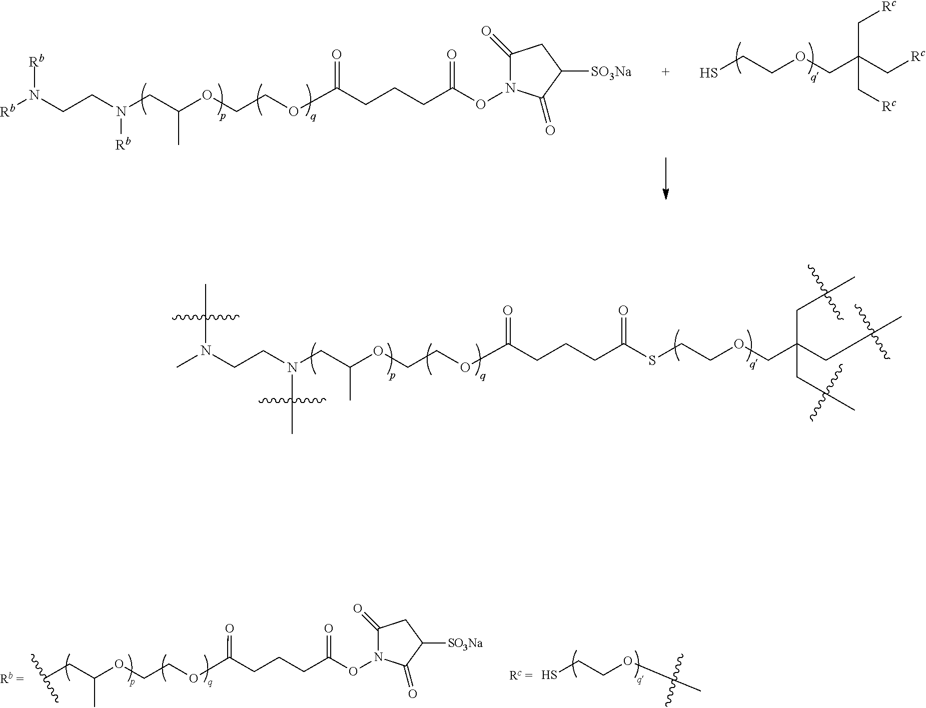

While the above is a complete description of the preferred embodiments of the invention, various alternatives, modifications, and equivalents may be used. Therefore, the above description should not be taken as limiting the scope of the invention which is defined by the appended claims.

1: A composition comprising first and second precursor molecules, wherein:
i) the first precursor molecule has a formula I:
A-[(C₃H₆O)ₘ—(C₂H₄O)ₙ—B]_{y} (I)
wherein m is an integer from 0 to 200;
n is an integer from 1 to 200;
y is 2 or greater;
A is a branch point; and
B comprises a nucleophilic functional group;
ii) the second precursor molecule has a formula II:
D-[(C₃H₆O)ₚ—(C₂H₄O)_{q}-E]_{z} (II)
wherein p and q are independently integers from 1 to 200;
z is 2 or greater;
D is a branch point; and
E comprises an electrophilic functional group substituted with a positively charged functional group, a negatively charged functional group, a zwitterionic functional group, a protonated or deprotonated version thereof, or a salt thereof.

2: The composition of claim 1, wherein y is 2 to 8 and z is 2 to 8.

3. (canceled)

4: The composition of claim 1, wherein m is 0.

5: The composition of claim 1, wherein m is an integer from 1 to 200.

6-8. (canceled)

9: The composition of claim 1, wherein the branch point A is selected from the group consisting of carbon, glycerol, pentaerythritol, dipentaerythritol, and ethylene diamine; and the branch point D is selected from the group consisting of carbon, glycerol, pentaerythritol, dipentaerythritol, and ethylene diamine.

10. (canceled)

11: The composition of claim 1, wherein B comprises a thiol group or an amino group.

12: The composition of claim 1, wherein E comprises an electrophilic functional group substituted with an acid moiety or an anion or salt thereof.

13: The composition of claim 12, wherein the acid moiety or anion or salt thereof is selected from the group consisting of SO₃H, SO₃⁻, SO₂H, SO₂⁻, CO₂H, CO₂⁻, PO₃H₂, PO₃⁻, OPO₃H₂, OPO₃⁻², and salts thereof.

14-15. (canceled)

16: The composition claim 1, wherein E is: 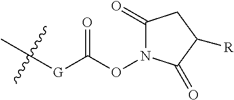
wherein G is absent or —CO(CH₂)ᵣ—;
r is an integer from 1 to 5; and
R is a positively charged functional group, a negatively charged functional group, a zwitterionic functional group, a protonated or deprotonated version thereof, or a salt thereof.

17: The composition of claim 16, wherein E is:

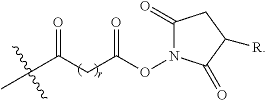

18. (canceled)

19: The composition of claim 16, wherein R is an acid moiety or an anion or salt thereof.

20: The composition of claim 19, wherein the acid moiety or anion or salt thereof is selected from the group consisting of SO₃H, SO₃⁻, SO₂H, SO₂⁻, CO₂H, CO₂⁻, PO₃H₂, PO₃⁻, OPO₃H₂, OPO₃⁻², and salts thereof.

21: The composition of claim 17, wherein r is 3 and R is SO₃H, SO₃⁻, or a salt thereof.

22-23.

24: The composition of claim 1, wherein the first precursor molecule has a formula: 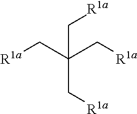
wherein each R^{1a} is 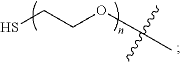
and
n is an integer from 1 to 200.

25: The composition of claim 1, wherein the first precursor molecule has a formula: 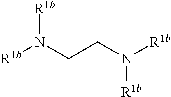
wherein each R^{1b} is 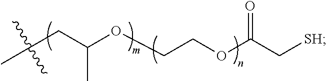
and
m and n are independently integers from 1 to 200.

26: The composition of claim 1, wherein the second precursor molecule has a formula: 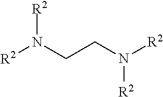
wherein each R² is 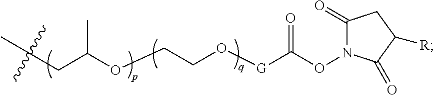
p and q are independently integers from 1 to 200;
G is absent or —CO(CH₂)ᵣ—;
r is an integer from 1 to 5; and
R is a positively charged functional group, a negatively charged functional group, a zwitterionic functional group, a protonated or deprotonated version thereof, or a salt thereof.

27: The composition of claim 26, wherein each R² is

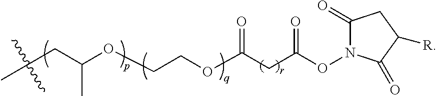

28. (canceled)

29: The composition of claim 26, wherein R is an acid moiety or an anion or salt thereof.

30: The composition of claim 29, wherein the acid moiety or anion or salt thereof is selected from the group consisting of SO₃H, SO₃⁻, SO₂H, SO₂⁻, CO₂H, CO₂⁻, PO₃H₂, PO₃⁻, OPO₃H₂, OPO₃⁻², and salts thereof.

31: The composition of claim 27, wherein r is 3 and R is SO₃H, SO₃⁻, or a salt thereof.

32. (canceled)

33: The composition of claim 1, wherein the composition is substantially free of poly(ethylene oxide) derivatives.

34: A dual compartment syringe comprising the composition of claim 1 loaded in a first compartment of the dual compartment syringe and a basic solution loaded in a second compartment of the dual compartment syringe.

35-37. (canceled)

38: A biomaterial formed by reacting the first and second precursor molecules of claim 1, wherein:
m is an integer from 1 to 200.

39: A biomaterial comprising a crosslinked polymeric network having a structure: 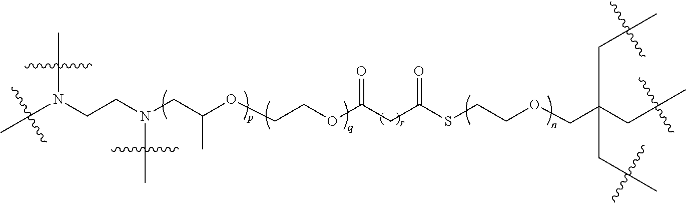
wherein n, p and q are independently integers from 1 to 200; and
r is an integer from 1 to 5; or
a biomaterial comprising a crosslinked polymeric network having a structure: 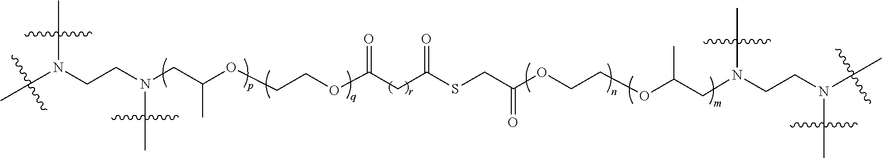
wherein m, n, p and q are independently integers from 1 to 200; and
r is an integer from 1 to 5.

40. (canceled)

41: A method of making a biomaterial comprising:
reacting first and second precursor molecules in the presence of a basic solution to form a biomaterial, wherein
i) the first precursor molecule has a formula I:
A-[(C₃H₆O)ₘ—(C₂H₄O)ₙ—B]_{y} (I)
wherein m is an integer from 0 to 200;
n is an integer from 1 to 200;
y is 2 or greater;
A is a branch point; and
B comprises a nucleophilic functional group;
ii) the second precursor molecule has a formula II:
D-[(C₃H₆O)ₚ—(C₂H₄O)_{q}—E]_{z} (II)
wherein p and q are independently integers from 1 to 200;
z is 2 or greater;
D is a branch point; and
E comprises an electrophilic functional group substituted with a positively charged functional group, a negatively charged functional group, a zwitterionic functional group, a protonated or deprotonated version thereof, or a salt thereof; and
the biomaterial is in the form of a crosslinked three dimensional network.

42-43. (canceled)

44: A method of sealing a tissue in a patient, comprising:
applying to a tissue in a patient the composition of claim 1.

45: A kit comprising:
the first and second precursor molecules of claim 1.

46-53. (canceled)

54: A single compartment syringe comprising the composition of claim 1.

55-56. (canceled)
